(19) United States
(12) Patent Application Publication
(10) Pub. No.: US 2026/0276645 A1
(43) Pub. Date: Sep. 17, 2026

(54) FLUORESCENCE POLARIZATION IMMUNOASSAY AND FLUORESCENTLY LABELED SUBSTANCE

(71) Applicant: Tianma Japan, Ltd., Kawasaki (JP)

(72) Inventors: Ayuko IMAI, Kawasaki (JP); Kenichi Hayashi, Kawasaki (JP); Koji Shigemura, Kawasaki (JP)

(73) Assignee: Tianma Japan, Ltd., Kawasaki (JP)

(21) Appl. No.: 19/553,679

(22) Filed: Mar. 2, 2026

(30) Foreign Application Priority Data
Mar. 17, 2025 (JP) 2025-042361

(51) Int. Cl. G01N 33/58; G01N 21/64; G01N 33/68

(52) U.S. Cl. CPC G01N 33/582; G01N 21/6428; G01N 33/6854

(57) ABSTRACT

A fluorescence polarization immunoassay uses a fluorescently labeled substance in which an antibody having binding ability to a measurement target substance is labeled with a fluorescent dye, to analyze the measurement target substance in a sample solution. The fluorescence polarization immunoassay includes a mixing step of mixing the sample solution and a solution containing the fluorescently labeled substance, and a measurement step of measuring a degree of fluorescence polarization of a mixed solution obtained in the mixing step. The fluorescently labeled substance has a specific property related to associability.

### CROSS-REFERENCE TO RELATED APPLICATIONS

This application claims the benefit of Japanese Patent Application No. 2025-42361, filed on Mar. 17, 2025, the entire disclosure of which is incorporated by reference herein.

### FIELD OF THE INVENTION

The present disclosure relates to a fluorescence polarization immunoassay and a fluorescently labeled substance.

### BACKGROUND OF THE INVENTION

One immunoassay that utilizes an antigen-antibody reaction is a measurement method called a fluorescence polarization immunoassay (FPIA), in which a measurement target substance is analyzed by fluorescence polarization measurement. In the FPIA, fluorescence intensity in a direction parallel to a polarization direction of excitation light and fluorescence intensity in a direction perpendicular to the polarization direction of the excitation light are each obtained, and the degree of bias of the fluorescence intensity in both directions is measured as the degree of fluorescence polarization. There are two types of FPIA: competitive and noncompetitive.

In a noncompetitive FPIA, a measurement target substance is caused to react with a fluorescently labeled antibody (tracer). The tracer not bound to the measurement target substance moves vigorously in solution, and randomly emits fluorescence even when irradiated with polarized excitation light. Meanwhile, the tracer bound to the measurement target substance is less likely to move and, as such, emits fluorescence biased in the polarization direction of the excitation light. The noncompetitive FPIA is thus suitable for analysis of a measurement target substance having a high molecular weight. In the noncompetitive FPIA, an increase in the amount of the measurement target substance in solution causes a larger amount of tracer to be bound to the measurement target substance. The binding between the tracer and the measurement target substance is an equilibrium reaction. Thus, in the noncompetitive FPIA, the relationship between the amount of the measurement target substance in solution and the degree of fluorescence polarization is represented by a sigmoid curve.

In the noncompetitive FPIA, the overall molecular volume of the tracer preferably changes significantly before and after the tracer is bond to the measurement target substance. Thus, the noncompetitive FPIA suitably uses a tracer having a low molecular weight. Unexamined Japanese Patent Application Publication No. 2021-173731 discloses a noncompetitive FPIA that uses, as a tracer, a fluorescently labeled substance in which a single domain antibody is labeled with a fluorescent dye. The single domain antibody disclosed in Unexamined Japanese Patent Application Publication No. 2021-173731 has an average mass of 12 kDa to 15 kDa, and is an antibody having a significantly lower molecular weight than conventional IgG antibodies. Unexamined Japanese Patent Application Publication No. 2021-173731 discloses that the use of the single domain antibody improves detection sensitivity, enabling measurement of a sample containing a measurement target substance at low concentration and measurement of a sample containing a measurement target substance having a high molecular weight.

### SUMMARY OF THE INVENTION

As a result of investigating a noncompetitive FPIA using a single domain antibody, the inventors of the present disclosure discovered that, contrary to conventional noncompetitive FPIAs, there are cases in which the degree of fluorescence polarization decreases as the amount of a measurement target substance in a sample solution increases. Further, the inventors of the present disclosure found that the aggregation state of a tracer affects this phenomenon, and completed the present disclosure.

That is, the present disclosure provides a fluorescence polarization immunoassay using a fluorescently labeled substance in which an antibody having binding ability to a measurement target substance is labeled with a fluorescent dye, to analyze the measurement target substance in a sample solution. The fluorescence polarization immunoassay includes:

a mixing step of mixing the sample solution and a solution containing the fluorescently labeled substance; and

a measurement step of measuring a degree of fluorescence polarization of a mixed solution obtained in the mixing step, wherein

the fluorescently labeled substance has at least one of the following properties:

a1) when a plurality of solutions containing the measurement target substance at mutually different concentrations are each mixed with a solution containing the fluorescently labeled substance, and a degree of fluorescence polarization of each of a plurality of resultant mixed solutions is measured, the degree of fluorescence polarization decreases as a concentration of the measurement target substance increases, within at least a portion of a concentration range; and

a2) the fluorescently labeled substance is capable of forming an aggregate in solution.

The present disclosure provides a fluorescence polarization immunoassay using a fluorescently labeled substance in which an antibody having binding ability to a measurement target substance is labeled with a fluorescent dye, to analyze the measurement target substance in a sample solution. The fluorescence polarization immunoassay includes:

a binding step of mixing the sample solution and a solution containing the fluorescently labeled substance, and thereby binding at least a portion of the fluorescently labeled substance in an aggregation state to the measurement target substance to be in a dispersion state; and

a measurement step of measuring a degree of fluorescence polarization of a mixed solution obtained in the binding step.

The present disclosure provides a fluorescently labeled substance in which an antibody is labeled with a fluorescent dye. The fluorescently labeled substance has at least one of the following properties:

b1) when a plurality of aqueous solutions containing an antigen at mutually different concentrations are each mixed with an aqueous solution containing the fluorescently labeled substance, and a degree of fluorescence polarization of each of a plurality of resultant mixed solutions is measured, the degree of fluorescence polarization decreases as a concentration of the antigen increases, within at least a portion of a concentration range; and

b2) the fluorescently labeled substance is capable of forming an aggregate in aqueous solution.

It is to be understood that both the foregoing general description and the following detailed description are exemplary and explanatory and are not restrictive of this disclosure.

### BRIEF DESCRIPTION OF DRAWINGS

A more complete understanding of this application can be obtained when the following detailed description is considered in conjunction with the following drawings, in which:

FIG. 1 is a schematic drawing illustrating the configuration of a fluorescence polarization immunoassay device 1;

FIG. 2 is a schematic drawing of a microdevice 23;

FIG. 3 is a drawing illustrating the relationship between the concentrations of ovalbumin standard solutions and the degrees of fluorescence polarization;

FIG. 4 is a drawing illustrating the relationship between the concentrations of β-lactoglobulin standard solutions and the degrees of fluorescence polarization, and the relationship between the concentrations of the ovalbumin standard solutions and the degrees of fluorescence polarization;

FIG. 5 is a drawing illustrating the relationship between the concentrations of blocking buffer solutions and the degrees of fluorescence polarization; and

FIG. 6 is a drawing illustrating an effect of the amount of a blocking agent on the relationship between the concentrations of the ovalbumin standard solutions and the degrees of fluorescence polarization.

### DETAILED DESCRIPTION OF THE INVENTION

A first embodiment of the present disclosure is a fluorescence polarization immunoassay using a fluorescently labeled substance in which an antibody having binding ability to a measurement target substance is labeled with a fluorescent dye, to analyze the measurement target substance in a sample solution. The fluorescence polarization immunoassay includes:

a mixing step of mixing the sample solution and a solution containing the fluorescently labeled substance; and

a measurement step of measuring a degree of fluorescence polarization of a mixed solution obtained in the mixing step.

The fluorescently labeled substance has at least one of the above-described properties a1 and a2.

### Measurement target substance

The measurement target substance is a substance to be measured in the fluorescence polarization immunoassay of the present disclosure. The measurement target substance is a substance at least a portion of which is recognized as an epitope by the antibody. Examples of the measurement target substance include biological substances, drugs, and viruses.

The biological substances are various components produced in the bodies of organisms, various components excreted from the bodies, the organisms themselves, and the like. The organisms may be animals or plants. Examples of the biological substances include: hormones that are physiologically active substances synthesized and secreted by endocrine organs such as the hypothalamus, pituitary gland, thyroid gland, parathyroid glands, adrenal glands, pancreas, and gonads; metabolites such as nucleic acids, uric acids, purines, C-reactive proteins (CRP), apolipoproteins, high-density lipoproteins (HDL), low-density lipoproteins (LDL), and glycated hemoglobins; shellfish and bacterial toxins such as mycotoxin, aflatoxin B1, and botulinum toxin A; plant-derived alkaloids such as morphine, atropine, quinine, and cocaine; and bacteria such as escherichia coli, streptococcus, bacillus, salmonella, and pseudomonas aeruginosa.

The drugs are substances to be administered to humans and animals for treatment or the like. The drugs may be intended for administration to plants. Examples of the drugs include: antibiotics such as chloramphenicol and cyclosporine; and agrochemicals such as bactericides, fungicides, insecticides, herbicides, rodenticides, and plant growth regulators.

The viruses are microscopic infectious structures that replicate themselves using the cells of other organisms. Examples of the viruses include influenza viruses, corona viruses, hepatitis B viruses, hepatitis A viruses, hepatitis C viruses, and AIDS viruses.

The mass of the measurement target substance is preferably 1.5×10³ Da to 1.0×10⁸ Da, and more preferably 1.0×10⁵ Da to 1.0×10⁸ Da.

### Fluorescently labeled substance

The fluorescently labeled substance used in the present disclosure is a substance in which the antibody having binding ability to the measurement target substance is labeled with the fluorescent dye.

The antibody constituting the fluorescently labeled substance is an antibody having binding ability to the measurement target substance. That is, the antibody constituting the fluorescently labeled substance is capable of recognizing at least a portion of the measurement target substance as an epitope, and being bound to the measurement target substance. The type of the antibody is not specifically limited, but is preferably an antibody having a low molecular weight. As described later, the fluorescently labeled substance used in the present disclosure has a property of associating in solution when not being bound to the measurement target substance. When the antibody constituting the fluorescently labeled substance is an antibody having a low molecular weight, the hydrophobicity or hydrophilicity of a molecule to be subsequently modified, such as the fluorescent dye, is more readily reflected in the fluorescently labeled substance. This facilitates obtaining a fluorescently labeled substance having the desired associability. In the present disclosure, the term "antibody having a low molecular weight" refers to an antibody fragment that includes a portion of a full-length antibody molecule and has binding ability to an antigen.

Examples of the antibody having a low molecular weight include variable domain of heavy chain of heavy chain (VHH) antibodies, single variable new antigen receptor domain (vNAR) antibodies, single chain variable fragments (scFv), and Fab fragments. Among these, the antibody having a low molecular weight is preferably a single domain antibody, such as a VHH antibody or a vNAR antibody.

The single domain antibody, such as a VHH antibody or a vNAR antibody, is an antibody composed solely of a variable region of a heavy chain antibody. The VHH antibody is an antibody composed solely of a variable region of a heavy chain antibody derived from camelid animals such as Bactrian camels, dromedary camels, llamas, alpacas, vicunas, and guanacos. The vNAR antibody is an antibody composed solely of a variable region of a heavy chain antibody derived from cartilaginous fish such as sharks and rays.

The single domain antibody can be prepared by preparing a heavy chain antibody that recognizes the measurement target substance as an antigen, and cutting out a portion of the heavy chain antibody. For example, a heavy chain antibody-producing animal is immunized with the measurement target substance as the antigen, and a heavy chain antibody to be bound to the antigen is selected from B cells of the immunized animal. Next, a variable region of a VHH antibody, a vNAR antibody, or the like obtained by cleaving the heavy chain antibody with an enzyme or the like can be used as the single domain antibody. The single domain antibody is not limited to an isolate from the heavy chain antibody. The single domain antibody can be prepared by genetic engineering to have specific binding ability to a specific substance by referring to the DNA sequences of conventionally known VHH antibodies and vNAR antibodies or by using an antibody library or the like. In this method, some amino acid residues may be replaced with other amino acid residues for the purpose of improving heat resistance, chemical resistance, pressure resistance, and the like to the extent that a binding property to the measurement target substance is not impaired. The single domain antibody may be prepared by decomposing a conventionally known Fab antibody or scFv antibody to extract one variable region.

The mass of the antibody constituting the fluorescently labeled substance is preferably 12 kDa to 50 kDa, more preferably 12 kDa to 25 kDa, and further preferably 12 kDa to 15 kDa.

The fluorescent dye constituting the fluorescently labeled substance is a dye that emits fluorescence. The term "fluorescence" in the present disclosure refers to light emission generated by irradiating light that excites electrons. Further, the term "fluorescent dye" refers to a dye that emits fluorescence. In phosphorescence, as in fluorescence, an atom absorbs energy and becomes excited. Thus, the fluorescent dye in the present disclosure also includes a dye that emits phosphorescence. When the fluorescent dye emits phosphorescence, the degree of fluorescence polarization may be measured based on phosphorescence instead of fluorescence.

Examples of the fluorescent dye that can be used in the present disclosure include: fluorescein compounds such as chlorotriazinyl aminofluorescein, 4'-aminomethylfluorescein, 5-aminomethylfluorescein, 6-aminomethylfluorescein, 6-carboxyfluorescein, 5-carboxyfluorescein, 5(6)-aminofluorescein, thioureafluorescein, and methoxytriazinylaminofluorescein; nitrobenzoxadiazole derivatives such as nitrobenzoxadiazole chloride; indolenine; dansyl derivatives such as dansyl; naphthalene derivatives such as dialkylaminonaphthalene and dialkylaminonaphthalenesulfonyl; pyrene derivatives such as N-(1-pyrenyl) maleimide, aminopyrene, pyrenebutanoic acid, and alkynylpyrene; metal complexes such as platinum, rhenium, ruthenium, osmium, and europium; rhodamine derivatives such as rhodamine B, rhodamine 6G, and rhodamine 6GP; and, as registered trademark or product names, Alexa Fluor series such as Alexa Fluor 488; BODIPY series; DY series; ATTO series; Dy Light series; Oyster series; HiLyte Fluor series; Pacific Blue; Marina Blue; Acridine, Edans; Coumarin; DANSYL; FAN; Oregon Green; Rhodamine Green-X; NBD-X; TET; JOE; Yakima Yellow; VIC; HEX; R6G; Cy3; TAMRA; Rhodamine Red-X; Redmond Red; ROX; Cal Red; Texas Red; LC Red 640; Cy5; Cy5.5; and LC Red 705. Ruthenium emits phosphorescence, and its life is 2,700 nanoseconds.

A preparation method of the fluorescently labeled substance is not specifically limited. For example, the fluorescently labeled substance can be prepared by causing the antibody to react with the fluorescent dye. The fluorescent dye generally has, within its molecules, functional groups such as an amino group, a carboxyl group, a halogen atom, and a nitro group. The antibody contains amino acid residues and, as such, the antibody and the fluorescent dye can be caused to react in accordance with a conventional method by utilizing the amino acid residues having functional groups. For example, the fluorescently labeled substance can be prepared by activating the functional groups of the fluorescent dye, and then mixing the fluorescent dye with the antibody to cause the fluorescent dye to react with the antibody for several hours at 4°C to 65°C. Further, when the single domain antibody or the like is produced by genetic engineering, an antibody containing, at a predetermined site, amino acid residues each having an amino group, a carboxyl group, a thiol group, and the like capable of reacting with a fluorescent dye may be prepared in consideration of the site at which the fluorescent dye is to be introduced, and be caused to react with the fluorescent dye having corresponding functional groups.

The number of fluorescent dye molecules to be bound to one antibody molecule can be arbitrarily selected. One or more fluorescent dye molecules are preferably bound to one antibody molecule, and two to five fluorescent dye molecules are more preferably bound to one antibody molecule.

The fluorescently labeled substance used in the present disclosure has at least one of the following properties related to associability:

a1) when a plurality of solutions containing the measurement target substance at mutually different concentrations are each mixed with a solution containing the fluorescently labeled substance, and the degree of fluorescence polarization of each of a plurality of resultant mixed solutions is measured, the degree of fluorescence polarization decreases as the concentration of the measurement target substance increases, within at least a portion of a concentration range; and

a2) the fluorescently labeled substance is capable of forming an aggregate in solution.

In the mixing of the solutions containing the measurement target substance and the solution containing the fluorescently labeled substance in the above-described property a1, each of these solutions may be a dilution of the solution containing the measurement target substance or a dilution of the solution containing the fluorescently labeled substance. In the above-described property a1, each of the mixed solutions the degrees of fluorescence polarization of which are measured may be a dilution of the mixed solution.

In a noncompetitive FPIA, the relationship between the amount of a measurement target substance in a sample solution and the degree of fluorescence polarization is represented by a sigmoid curve. Therefore, a reaction system having the property that the degree of fluorescence polarization decreases as the concentration of a measurement target substance increases is different from reaction systems of conventional noncompetitive FPIAs. As described below, the phenomenon in which the degree of fluorescence polarization decreases as the concentration of a measurement target substance increases can be rationally understood by considering associability of a fluorescently labeled substance.

When a fluorescently labeled substance in a solution associates to form aggregates, the movement of the fluorescently labeled substance constituting the aggregates becomes restricted. Thus, the degree of fluorescence polarization of the solution becomes high. When a measurement target substance is added to the solution containing the aggregates of the fluorescently labeled substance, a portion of the fluorescently labeled substance constituting the aggregates is bound to the measurement target substance to form measurement target substance–fluorescently labeled substance complexes. When the measurement target substance has high affinity for the solution and the measurement target substance–fluorescently labeled substance complexes have a lower molecular weight than the aggregates of the fluorescently labeled substance, the number of vigorously moving molecules (the measurement target substance–fluorescently labeled substance complexes) increases as the concentration of the measurement target substance increases. Thus, the degree of fluorescence polarization of the solution becomes low. The above-described property a1 is based on this phenomenon.

A blocking agent is also considered to have an effect of dispersing the fluorescently labeled substance constituting the aggregates. Therefore, as a preliminary experiment prior to investigating the above-described property a1, the relationship between the concentration of the blocking agent and the degree of fluorescence polarization of the solution may be examined.

The blocking agent is not specifically limited provided that the blocking agent has the effect of dispersing the fluorescently labeled substance constituting the aggregates. Examples of the blocking agent include biological substances such as bovine serum albumin (BSA), casein (α-casein, β-casein, and γ-casein), and gelatin.

The above-described property a2 can be investigated by, for example, multi-angle light scattering (MALS). The property a2 may be investigated by preparing a solution containing a higher concentration of the fluorescently labeled substance and a solution containing a larger amount of poor solvent, and observing these solutions.

When the properties a1 and a2 are observed in a solution, the FPIA of the present disclosure that operates on a principle different from that of conventional noncompetitive FPIAs can be performed by preparing a solution having components identical or similar to those of the above-described solution in the mixing step, and using the resultant mixed solution as a measurement solution in the measurement step. The term "measurement solution" in the present disclosure refers to a solution to be irradiated with excitation light. For example, when a mixed solution of a solution containing the measurement target substance and a solution containing the fluorescently labeled substance is irradiated with excitation light, the mixed solution is the measurement solution. When the diluted mixed solution is irradiated with excitation light, the diluted mixed solution is the measurement solution.

When the measurement target substance is a biological substance or the like, the measurement target substance is often a water-soluble substance, and the sample solution or measurement solution is generally an aqueous solution. A fluorescently labeled substance having at least one of properties b1 and b2 described later exhibits a hydrophobic property and readily associates in aqueous solution. Thus, when the measurement solution is an aqueous solution, the fluorescently labeled substance having at least one of the properties b1 and b2 is suitably used.

The term "aqueous solution" in the present disclosure refers to a solution in which the primary solvent is water such as pure water, purified water, physiological saline, or various buffer solutions. The aqueous solution may also contain a water-miscible organic solvent such as methanol or ethanol, provided that the solvent does not adversely affect the effects of the present disclosure. The content of the water-miscible organic solvent is generally 10% by mass or less, and preferably 5% by mass or less.

### Fluorescence polarization immunoassay

The fluorescence polarization immunoassay of the present disclosure includes:

a mixing step of mixing the sample solution and a solution containing the fluorescently labeled substance; and

a measuring step of measuring a degree of fluorescence polarization of a mixed solution obtained in the mixing.

The mixing step is a step of mixing the sample solution and the solution containing the fluorescently labeled substance. The sample solution is a solution containing the measurement target substance. When the measurement target substance is a biological substance or the like, an aqueous solution is generally used as the sample solution. In this case, an aqueous solution is also preferably used as the solution containing the fluorescently labeled substance.

The sample solution and the solution containing the fluorescently labeled substance in the mixing step may be dilutions thereof. Examples of a diluent include pure water, purified water, physiological saline, and various buffer solutions.

The measurement step is a step of measuring the degree of fluorescence polarization of the mixed solution obtained in the mixing step. The mixed solution may contain a blocking agent. Examples of the blocking agent include biological substances such as bovine serum albumin (BSA), casein (α-casein, β-casein, and γ-casein), and gelatin. In some cases, the mixed solution containing the blocking agent allows measurement of the degree of fluorescence polarization under more appropriate conditions. In measurement of the degree of fluorescence polarization of the mixed solution, the mixed solution itself may be used as the measurement solution, and a dilution of the mixed solution may be used as the measurement solution.

A measurement device or measurement equipment used in the measurement step is not specifically limited, but the degree of fluorescence polarization can be efficiently measured by using a microdevice.

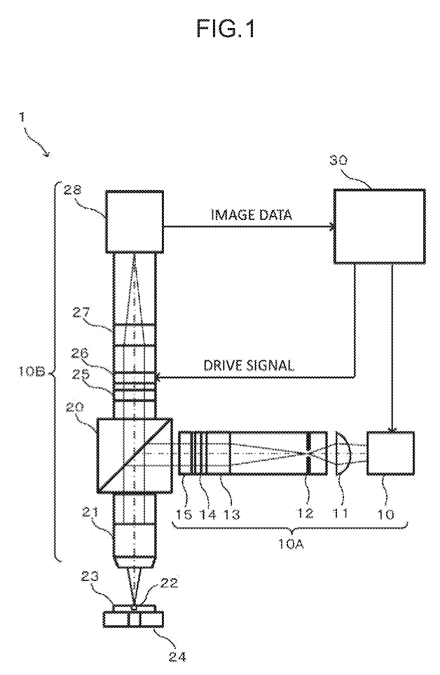

FIG. 1 illustrates an example of a fluorescence polarization immunoassay device that uses a microdevice. The fluorescence polarization immunoassay device 1 includes a light source 10, a condenser lens 11, an iris 12, a collimator 13, a polarization element 14, an excitation light filter 15, and a dichroic mirror 20. The fluorescence polarization immunoassay device 1 includes an objective lens 21, a microdevice 23 on which a sample 22 is placed, a stage 24, an absorption filter 25, a polarized light adjustment element 26, an imaging lens 27, an imaging element 28, and a controller 30.

In one example, the light source 10 is implemented as a light emitting diode, and emits excitation light of a wavelength that excites fluorescence of the sample (for example, blue light of a central wavelength of 470 nm). The excitation light from the light source 10 is focused by the condenser lens 11 and passes through the iris 12. The iris 12 reduces the intrusion of external light other than the excitation light.

The excitation light that has passed through the iris 12 is converted to parallel light by the collimator 13, and enters the polarization element 14. The polarization element 14 is, for example, a polarizing plate, a polarizing beam splitter, or a liquid crystal cell and, in this case, is a polarizing plate. The polarization element 14 allows light linearly polarized in a specific direction to pass through. The linearly polarized excitation light from the polarization element 14 passes through the excitation light filter 15. The excitation light filter 15 is a filter that selects a wavelength range including the wavelength of the excitation light, and reduces light of a wavelength different from that of the excitation light from the polarization element 14. The dichroic mirror 20 reflects, toward the objective lens 21, the excitation light that has passed through the excitation light filter 15.

The objective lens 21 focuses the linearly polarized excitation light reflected by the dichroic mirror 20 on the sample 22 accommodated in the microdevice 23 on the stage 24. The sample 22 generates fluorescence of a specific wavelength (for example, green light) in accordance with the linearly polarized excitation light from the objective lens 21. The fluorescence becomes parallel light at the objective lens 21, and passes through the dichroic mirror 20 and the absorption filter 25. The dichroic mirror 20 selectively allows light of a specific wavelength range including the wavelength of the fluorescence from the sample 22 to pass through, and reflects other light. The absorption filter 25 is a filter that selects a wavelength range including the wavelength of the fluorescence from the sample 22, and reduces light other than the fluorescence.

The fluorescence that has passed through the absorption filter 25 enters the polarized light adjustment element 26. In one example, the polarized light adjustment element 26 is implemented as a polarizing plate, a polarizing beam splitter, or a liquid crystal cell. The polarized light adjustment element 26 may be implemented as a polarization filter in a polarization camera. The polarization camera is an imaging device that includes the polarization filter mounted on a sensor and thereby acquires polarization information of a subject. In the following description, the polarized light adjustment element 26 is a liquid crystal cell controlled by a drive signal (applied voltage). The polarized light adjustment element 26 can adjust transmitted light intensities of linearly polarized components. Specifically, the polarized light adjustment element 26 can adjust transmitted light intensities of: linearly polarized light parallel to the polarization direction of the excitation light; linearly polarized light perpendicular to the polarization direction of the excitation light; and light polarized in a direction corresponding to the drive signal described later.

The linearly polarized fluorescence that has passed through the polarized light adjustment element 26 enters an imaging plane of the imaging element 28 via the imaging lens 27. The surface of the sample 22 and the imaging plane of the imaging element 28 are in an imaging relationship. The imaging element 28 includes, for example, a charge-coupled device (CCD) or a complementary metal-oxide-semiconductor (CMOS) image sensor that includes a plurality of pixels. The imaging element 28 generates image data corresponding to the intensity of the fluorescence generated by the sample 22, and sends the generated image data to the controller 30.

The controller 30 performs overall control of the entire fluorescence polarization immunoassay device 1. Specifically, the controller 30 controls the light source 10, the polarized light adjustment element 26, and the imaging element 28. The controller 30 acquires fluorescence images captured by the imaging element 28.

For example, during measurement operation, the controller 30 causes the light source 10 to emit the excitation light on the sample 22. The controller 30 uses a digital-to-analog (DA) converter (not illustrated) to output the drive signal to the polarized light adjustment element 26. By outputting the drive signal to the polarized light adjustment element 26, the controller 30 can control polarized light components of the fluorescence that pass through the polarized light adjustment element 26.

In one example, the polarized light adjustment element 26 includes two transparent substrates that face each other, a transparent electrode disposed on opposing surfaces of the substrates, liquid crystal material sealed between the substrates, and a polarizing plate disposed on an outer surface on the imaging device side (exit side or downstream side) of the polarized light adjustment element 26. The configuration of the polarized light adjustment element 26 may be determined as desired provided that it is possible to adjust the polarized light components of the fluorescence that pass through the polarized light adjustment element 26.

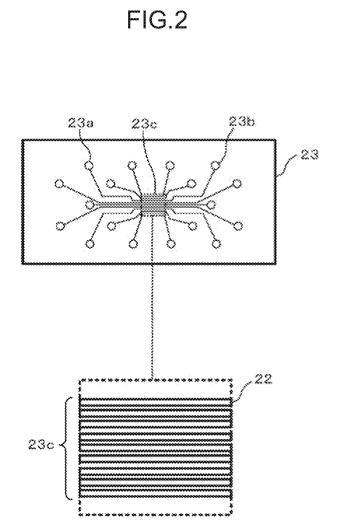

As illustrated in FIG. 2, the microdevice 23 includes a plurality of channels 23c that each include one end connected to an injection port 23a and another end connected to a discharge port 23b. The measurement solution can be individually supplied as the sample 22 to the plurality of channels 23c. The channels 23c and the imaging plane of the imaging element 28 are in an imaging relationship and, as such, if the plurality of channels 23c are captured by the imaging element 28, fluorescence images of the plurality of samples 22 supplied to the plurality of channels 23c can be obtained at once, and the degrees of polarization of the respective samples 22 can be measured at once. This measurement is performed on the basis of the fluorescence intensity of a region of interest (ROI) of image data corresponding to each of the channels 23c in the fluorescence images.

When the fluorescence polarization immunoassay of the present disclosure is performed, preliminary experiments may be conducted in advance to examine solubility and the like of the measurement target substance and the fluorescently labeled substance in a solvent intended for use in preparing the measurement solution. In some cases, the fluorescence polarization immunoassay of the present disclosure can be more efficiently performed by conducting the preliminary experiments.

The fluorescence polarization immunoassay of the present disclosure is based on a state change between the aggregates of the fluorescently labeled substance and the measurement target substance–fluorescently labeled substance complexes, unlike conventional noncompetitive FPIAs based on a state change between a free fluorescently labeled substance and measurement target substance–fluorescently labeled substance complexes. Thus, in the fluorescence polarization immunoassay of the present disclosure, a small amount of the measurement target substance can be detected. For example, the lower limit of detection in measurement of ovalbumin described in the examples is 0.3 ppm.

A second embodiment of the present disclosure is a fluorescence polarization immunoassay using a fluorescently labeled substance in which an antibody having binding ability to a measurement target substance is labeled with a fluorescent dye, to analyze the measurement target substance in a sample solution. The fluorescence polarization immunoassay includes:

a binding step of mixing the sample solution and a solution containing the fluorescently labeled substance, and thereby binding at least a portion of the fluorescently labeled substance in an aggregation state to the measurement target substance to be in a dispersion state; and

a measurement step of measuring a degree of fluorescence polarization of a mixed solution obtained in the binding step.

Examples of the measurement target substance in the second embodiment include those exemplified in the description of the fluorescence polarization immunoassay in the first embodiment. The fluorescently labeled substance used in the second embodiment is a substance in which the antibody having binding ability to the measurement target substance is labeled with the fluorescent dye. Examples of the antibody and the fluorescent dye include those exemplified in the description of the fluorescence polarization immunoassay in the first embodiment.

In the binding step in the second embodiment, the same operations as those in the mixing step of the fluorescence polarization immunoassay in the first embodiment are performed. At an early stage of the binding step, the fluorescently labeled substance associates to form aggregates in the mixed solution. At least a portion of the fluorescently labeled substance constituting the aggregates is then bound to the measurement target substance and dissociates from the aggregates.

In the measurement step in the second embodiment, the degree of fluorescence polarization of the mixed solution obtained in the binding step is measured. In this mixed solution, at least a portion of the fluorescently labeled substance constitutes measurement target substance-fluorescently labeled substance complexes and is dispersed in the mixed solution. The measurement step in the second embodiment can be performed in the same manner as the measurement step of the fluorescence polarization immunoassay in the first embodiment.

As with the fluorescence polarization immunoassay in the first embodiment, the fluorescence polarization immunoassay in the second embodiment is based on a state change between the aggregates of the fluorescently labeled substance and the measurement target substance–fluorescently labeled substance complexes. Thus, in the fluorescence polarization immunoassay in the second embodiment, a small amount of the measurement target substance can be detected.

A third embodiment of the present disclosure is a fluorescently labeled substance in which an antibody is labeled with a fluorescent dye. The fluorescently labeled substance in the third embodiment is a fluorescently labeled substance in which the antibody is labeled with the fluorescent dye. Examples of the antibody and the fluorescent dye constituting the fluorescently labeled substance include those exemplified in the description of the fluorescence polarization immunoassay in the first embodiment. Further, the fluorescently labeled substance in the third embodiment has at least one of the properties b1 and b2 described below.

b1) When a plurality of aqueous solutions containing an antigen at mutually different concentrations are each mixed with an aqueous solution containing the fluorescently labeled substance, and the degree of fluorescence polarization of each of a plurality of resultant mixed solutions is measured, the degree of fluorescence polarization decreases as the concentration of the antigen increases, within at least a portion of a concentration range.

b2) The fluorescently labeled substance is capable of forming an aggregate in aqueous solution.

The properties b1 and b2 are the same as the properties a1 and a2, except that the solutions are limited to aqueous solutions. That is, the fluorescently labeled substance in the third embodiment has a property of readily associating in aqueous solution. Thus, in the fluorescence polarization immunoassay in the first embodiment, when the measurement solution is an aqueous solution, the fluorescently labeled substance in the third embodiment is suitably used.

The antibody constituting the fluorescently labeled substance in the third embodiment preferably contains a large number of hydrophobic amino acid residues. Examples of the hydrophobic amino acid include glycine, alanine, valine, leucine, isoleucine, methionine, proline, phenylalanine, and tryptophan.

The antibody constituting the fluorescently labeled substance in the third embodiment may have hydrophobic groups introduced by reactions involving terminal amino groups, terminal carboxyl groups, functional groups in side chains, or the like. The introduction of hydrophobic groups can be performed in the same manner as the labeling method using the fluorescent dye.

The fluorescent dye constituting the fluorescently labeled substance in the third embodiment is preferably hydrophobic. Examples of the hydrophobic fluorescent dye include fluorescent dyes containing aromatic rings within molecules and fluorescent dyes having few hydrophilic groups such as carboxyl groups or amino groups.

Thus, the fluorescently labeled substance in the third embodiment can be efficiently prepared by appropriately adjusting the molecular weight of the antibody, the property (hydrophobicity) that the antibody inherently has, and the property (hydrophobicity) that is newly introduced to the antibody by the fluorescent dye or the like.

The fluorescently labeled substance in the third embodiment has at least one of the properties b1 and b2, and readily associates in aqueous solution. The fluorescently labeled substance in the third embodiment allows a high-sensitivity FPIA using an aqueous solution as the measurement solution to be performed. Thus, a small amount of the measurement target substance in sample solution can be analyzed by using this fluorescently labeled substance.

### Examples

### Example 1

An anti-ovalbumin VHH antibody (manufactured by Creative Biolabs) was modified with the Alexa Fluor 546 fluorescent dye using a protein labeling reagent (manufactured by Thermo Fisher Scientific) to prepare a fluorescently labeled anti-ovalbumin VHH antibody.

### Example 2

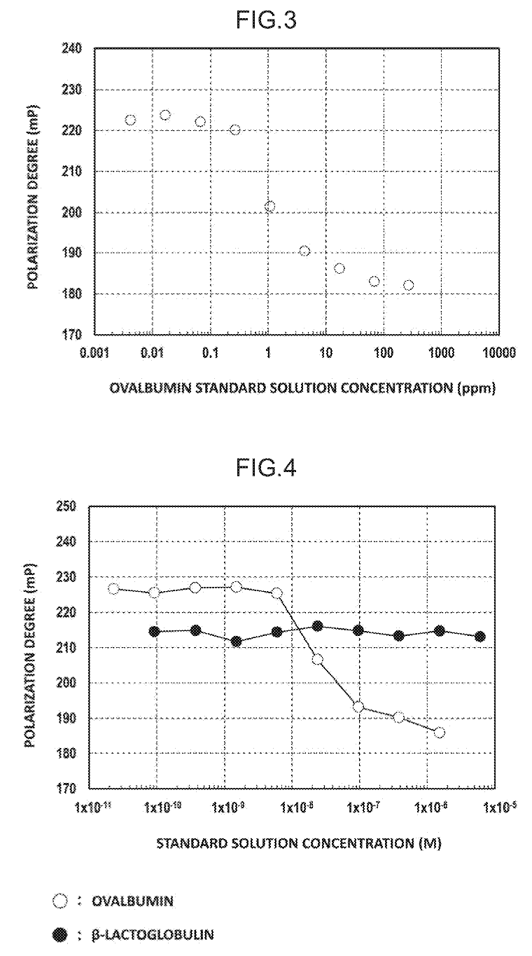

Ovalbumin (manufactured by Sigma-Aldrich) was dissolved in phosphate-buffered saline (PBS) (manufactured by FUJIFILM Wako Pure Chemical Corporation) and further diluted with PBS to prepare nine-level ovalbumin standard solutions at 275 ppm, 69 ppm, 17 ppm, 4.3 ppm, 1.1 ppm, 0.27 ppm, 0.067 ppm, 0.017 ppm, and 0.0042 ppm. Next, the fluorescently labeled anti-ovalbumin VHH antibody obtained in Example 1 was diluted using a solution prepared by diluting StartingBlock Blocking Buffer (manufactured by Thermo Fisher Scientific) fivefold with PBS to prepare a 30 nM fluorescently labeled anti-ovalbumin VHH antibody solution. Each of the nine-level 50 µL ovalbumin standard solutions was mixed with the 50 µL fluorescently labeled anti-ovalbumin VHH antibody solution to prepare nine-level 100 µL samples for creating an ovalbumin standard solution curve. The samples for creating an ovalbumin standard solution curve were allowed to rest in a light shielded environment at room temperature for one hour, and the degrees of fluorescence polarization were then measured. The results are illustrated in FIG. 3. It can be seen that, in the concentration range of 0.1 ppm to 10 ppm for the ovalbumin standard solutions, the degree of fluorescence polarization decreases sharply as the concentration increases.

### Comparative Example 1

β-lactoglobulin (manufactured by Sigma-Aldrich) was dissolved in phosphate-buffered saline (PBS) (manufactured by FUJIFILM Wako Pure Chemical Corporation) and further diluted with PBS to prepare nine-level β-lactoglobulin standard solutions at 6,110 nM, 1,530 nM, 382 nM, 96 nM, 24 nM, 6.0 nM, 1.5 nM, 0.37 nM, and 0.093 nM. Next, the fluorescently labeled anti-ovalbumin VHH antibody obtained in Example 1 was diluted using a solution prepared by diluting StartingBlock Blocking Buffer (manufactured by Thermo Fisher Scientific) fivefold with PBS to prepare a 30 nM fluorescently labeled anti-ovalbumin VHH antibody solution. Each of the nine-level 50 µL β-lactoglobulin standard solutions was mixed with the 50 µL fluorescently labeled anti-ovalbumin VHH antibody solution to prepare nine-level 100 µL samples for creating a β-lactoglobulin standard solution curve. The samples for creating a β-lactoglobulin standard solution curve were allowed to rest in a light shielded environment at room temperature for one hour, and the degrees of fluorescence polarization were then measured. The results are illustrated in FIG. 4. FIG. 4 also illustrates the measurement results obtained using ovalbumin standard solutions (at 1,530 nM, 382 nM, 96 nM, 24 nM, 6.0 nM, 1.5 nM, 0.37 nM, 0.093 nM, and 0.023 nM). For the ovalbumin standard solutions, changes in the degree of fluorescence polarization similar to those observed in Example 1 were observed. For the β-lactoglobulin standard solutions, however, it can be seen that changes in the concentration do not affect the degree of fluorescence polarization.

### Reference Example 1

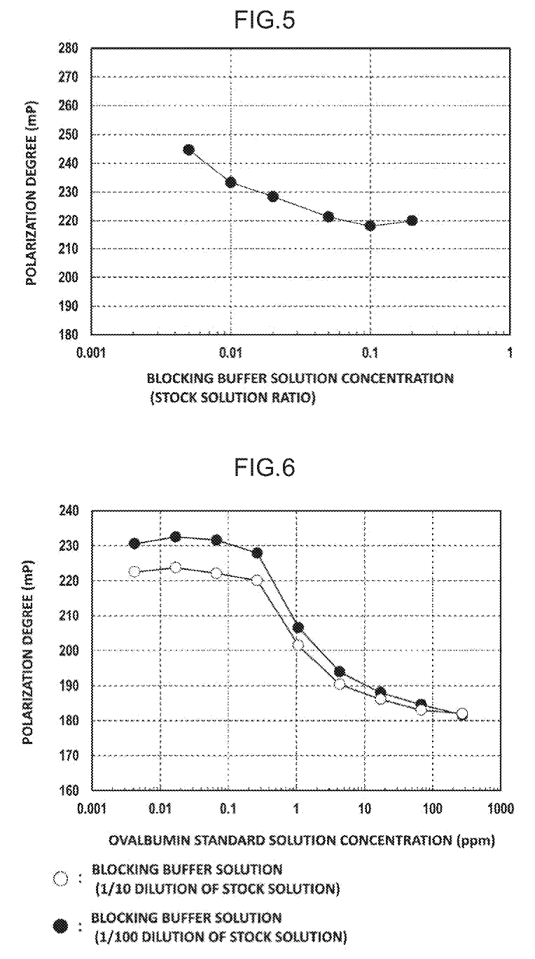

StartingBlock Blocking Buffer (manufactured by Thermo Fisher Scientific) was diluted with PBS fivefold, 10-fold, 20-fold, 50-fold, 100-fold, and 200-fold to prepare six-level StartingBlock Blocking Buffer solutions in which the ratio of the StartingBlock Blocking Buffer stock to the solution was 0.2 (–), 0.1 (–), 0.05 (–), 0.02 (–), 0.01 (–), and 0.005 (–), respectively. The fluorescently labeled anti-ovalbumin VHH antibody obtained in Example 1 was diluted with the six-level StartingBlock Blocking Buffer solutions such that the concentration of the fluorescently labeled anti-ovalbumin VHH antibody becomes 15 nM, to prepare six-level fluorescently labeled anti-ovalbumin VHH antibody solutions. The degrees of fluorescence polarization of the fluorescently labeled anti-ovalbumin VHH antibody solutions were measured. The results are illustrated in FIG. 5. In can be seen that the degree of fluorescence polarization decreases as the concentration of the blocking buffer solution increases.

### Example 3

Ovalbumin (manufactured by Sigma-Aldrich) was dissolved in phosphate-buffered saline (PBS) (manufactured by FUJIFILM Wako Pure Chemical Corporation) and further diluted with PBS to prepare nine-level ovalbumin standard solutions at 275 ppm, 69 ppm, 17 ppm, 4.3 ppm, 1.1 ppm, 0.27 ppm, 0.067 ppm, 0.017 ppm, and 0.0042 ppm. Next, the fluorescently labeled anti-ovalbumin VHH antibody obtained in Example 1 was diluted using a solution prepared by diluting StartingBlock Blocking Buffer (manufactured by Thermo Fisher Scientific) 50-fold with PBS to prepare a 30 nM fluorescently labeled anti-ovalbumin VHH antibody solution. Each of the nine-level 50 µL ovalbumin standard solutions was mixed with the 50 µL fluorescently labeled anti-ovalbumin VHH antibody solution to prepare nine-level 100 µL samples for creating an ovalbumin standard solution curve. The concentration of the StartingBlock Blocking Buffer in the samples for creating an ovalbumin standard solution curve is 1/100 of the stock solution. The samples for creating an ovalbumin standard solution curve were allowed to rest in a light shielded environment at room temperature for one hour, and the degrees of fluorescence polarization were then measured. The results are illustrated as those of the blocking buffer solution (1/100 dilution of the stock solution) in FIG. 6. FIG. 6 also illustrates the results obtained in Example 2 as those of the blocking buffer solution (1/10 dilution of the stock solution). In Example 2, the fluorescently labeled anti-ovalbumin VHH antibody obtained in Example 1 was diluted using the solution prepared by diluting StartingBlock Blocking Buffer fivefold with PBS. Therefore, the concentration of the StartingBlock Blocking Buffer in the samples for creating an ovalbumin standard solution curve in Example 2 is 1/10 of the stock solution. As illustrated in FIG. 6, the graph representing the relationship between the concentrations of the ovalbumin standard solutions and the degrees of polarization can be shifted by adjusting the amount of the blocking agent. Thus, an appropriate measurement environment can be established by using the blocking agent.

The foregoing describes some example embodiments for explanatory purposes. Although the foregoing discussion has presented specific embodiments, persons skilled in the art will recognize that changes may be made in form and detail without departing from the broader spirit and scope of the invention. Accordingly, the specification and drawings are to be regarded in an illustrative rather than a restrictive sense. This detailed description, therefore, is not to be taken in a limiting sense, and the scope of the invention is defined only by the included claims, along with the full range of equivalents to which such claims are entitled.

1. A fluorescence polarization immunoassay using a fluorescently labeled substance in which an antibody having binding ability to a measurement target substance is labeled with a fluorescent dye, to analyze the measurement target substance in a sample solution, the fluorescence polarization immunoassay comprising:
a mixing step of mixing the sample solution and a solution containing the fluorescently labeled substance; and
a measurement step of measuring a degree of fluorescence polarization of a mixed solution obtained in the mixing step, wherein
the fluorescently labeled substance has at least one of the following properties:
a1) when a plurality of solutions containing the measurement target substance at mutually different concentrations are each mixed with a solution containing the fluorescently labeled substance, and a degree of fluorescence polarization of each of a plurality of resultant mixed solutions is measured, the degree of fluorescence polarization decreases as a concentration of the measurement target substance increases, within at least a portion of a concentration range; and
a2) the fluorescently labeled substance is capable of forming an aggregate in solution.

2. A fluorescence polarization immunoassay using a fluorescently labeled substance in which an antibody having binding ability to a measurement target substance is labeled with a fluorescent dye, to analyze the measurement target substance in a sample solution, the fluorescence polarization immunoassay comprising:
a binding step of mixing the sample solution and a solution containing the fluorescently labeled substance, and thereby binding at least a portion of the fluorescently labeled substance in an aggregation state to the measurement target substance to be in a dispersion state; and
a measurement step of measuring a degree of fluorescence polarization of a mixed solution obtained in the binding step.

3. The fluorescence polarization immunoassay according to claim 1, wherein the measurement target substance is a biological substance, a drug, or a virus.

4. The fluorescence polarization immunoassay according to claim 1, wherein the measurement target substance has a mass of 1.5×10³ Da to 1.0×10⁸ Da.

5. The fluorescence polarization immunoassay according to claim 1, wherein the antibody is a VHH antibody or a vNAR antibody.

6. The fluorescence polarization immunoassay according to claim 1, wherein the antibody has a mass of 12 kDa to 50 kDa.

7. The fluorescence polarization immunoassay according to claim 1, wherein the measurement target substance is a water-soluble substance, and the sample solution is an aqueous solution.

8. The fluorescence polarization immunoassay according to claim 1, wherein the mixed solution contains a blocking agent.

9. The fluorescence polarization immunoassay according to claim 2, wherein the measurement target substance is a biological substance, a drug, or a virus.

10. The fluorescence polarization immunoassay according to claim 2, wherein the measurement target substance has a mass of 1.5×10³ Da to 1.0×10⁸ Da.

11. The fluorescence polarization immunoassay according to claim 2, wherein the antibody is a VHH antibody or a vNAR antibody.

12. The fluorescence polarization immunoassay according to claim 2, wherein the antibody has a mass of 12 kDa to 50 kDa.

13. The fluorescence polarization immunoassay according to claim 2, wherein the measurement target substance is a water-soluble substance, and the sample solution is an aqueous solution.

14. The fluorescence polarization immunoassay according to claim 2, wherein the mixed solution contains a blocking agent.

15. A fluorescently labeled substance in which an antibody is labeled with a fluorescent dye, the fluorescently labeled substance comprising at least one of the following properties:
b1) when a plurality of aqueous solutions containing an antigen at mutually different concentrations are each mixed with an aqueous solution containing the fluorescently labeled substance, and a degree of fluorescence polarization of each of a plurality of resultant mixed solutions is measured, the degree of fluorescence polarization decreases as a concentration of the antigen increases, within at least a portion of a concentration range; and
b2) the fluorescently labeled substance is capable of forming an aggregate in aqueous solution.

16. The fluorescently labeled substance according to claim 15, wherein the antibody contains a hydrophobic amino acid residue.

17. The fluorescently labeled substance according to claim 15, wherein the fluorescent dye contains an aromatic ring.
